(19) United States
(12) Patent Application Publication
(10) Pub. No.: US 2020/0051695 A1
(43) Pub. Date: Feb. 13, 2020

(54) TIME-SENSITIVE RISK MODEL CALCULATION

(71) Applicant: KONINKLIJKE PHILIPS N.V., EINDHOVEN (NL)

(72) Inventors: Merlijn Sevenster, Haarlem (NL); Thomas Andre Forsberg, Hayward, CA (US)

(21) Appl. No.: 16/339,895

(22) PCT Filed: Oct. 27, 2017
(86) PCT No.: PCT/EP2017/077701

§ 371 Date: Apr. 5, 2019

Related U.S. Application Data
(60) Provisional application No. 62/414,048, filed on Oct. 28, 2016.

(51) Int. Cl. G16H 50/30; G06N 5/04; G16H 10/60

(52) U.S. Cl. CPC G16H 50/30; G16H 10/60; G06N 5/04

(57) ABSTRACT

A system and method for alerting a user of a new risk score. The system includes a processor that receives a medical report for a patient, extract data from the medical report, computes a risk score based at least in part on the extracted data, compares the risk score to a previous risk score of the patient and determines when a change between the risk score and the previous risk score exceeds a predetermined threshold. The system further includes a display that displays an alert when the change exceeds the predetermined threshold.

### BACKGROUND

Risk models have been developed to predict the risk of a certain adverse clinical event based on input parameters. For example, an Acute Kidney Injury (AKI) model predicts the risk of nephropathy caused by a contrast used during cardiology intervention. This provides a patient and a patient's physician with increased awareness of the risks involved with medical procedures. The parameters utilized by the AKI model may include hypotension, pre-interventional use of a balloon pump, chronic heart failure, age, anemia, diabetes, contrast medium volume, epidermal growth factor receptor (eGFR), etc. The AKI model may use any or all of these parameters to produce a percentage risk score or a risk score category, such as low/medium/high. The percentage risk score or the risk score category may then be consulted by, for example, the patient's physician, for purposes such as intervention planning (e.g., whether to perform the cardiology intervention).

However, between the scheduling of an interventional procedure (or any determined treatment) and the occurrence of the interventional procedure and/or treatment, new information, such as information from a newly performed lab test, may become available. The new information may refine the risk score, allow for completion of the risk score if the risk score was generated based on only a partial amount of the necessary parameters, or contradict a value of a parameter utilized in the generation of the risk score. Each of these events or changes to the risk score may be used to change the course of treatment of the patient.

If a user of the risk model, such as the patient's physician, a specialist (e.g., cardiologist), administrative staff, etc., does not receive indications or alerts indicating that the new information is available, the patient's treatment regimen may not be altered, resulting in sub-optimum healthcare. In current systems, there is no mechanism to notify the user when the risk score changes due to an updated parameter value and thus, the user may be operating based on an outdated and incorrect risk score, thereby possibly endangering the patient or treating the patient in a suboptimal manner.

### SUMMARY

In one exemplary embodiment, a system is provided for alerting a user of a new risk score. The system includes a processor configured to receive a medical report for a patient, extract data from the medical report, compute a risk score based at least in part on the extracted data, compare the risk score to a previous risk score of the patient and determine when a change between the risk score and the previous risk score exceeds a predetermined threshold. The system further includes a display that displays an alert when the change exceeds the predetermined threshold.

In another exemplary embodiment, a method is described for alerting a user of a new risk score. The method includes receiving a medical report for a patient, extracting data from the medical report, computing a risk score based at least in part on the extracted data, comparing the risk score to a previous risk score of the patient, determining when a change between the risk score and the previous risk score exceeds a predetermined threshold and displaying an alert when the change exceeds the predetermined threshold.

In another exemplary embodiment, a non-transitory computer readable storage medium with an executable program stored thereon is described. The program, when executed, instructs a processor to perform actions that include receiving a medical report for a patient, extracting data from the medical report, computing a risk score based at least in part on the extracted data, comparing the risk score to a previous risk score of the patient, determining when a change between the risk score and the previous risk score exceeds a predetermined threshold and displaying an alert when the change exceeds the predetermined threshold.

### BRIEF DESCRIPTION

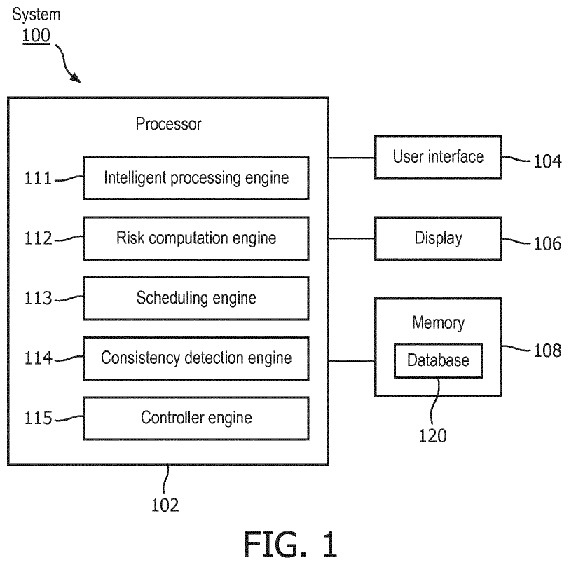

FIG. 1 shows a schematic drawing of a system according to an exemplary embodiment.

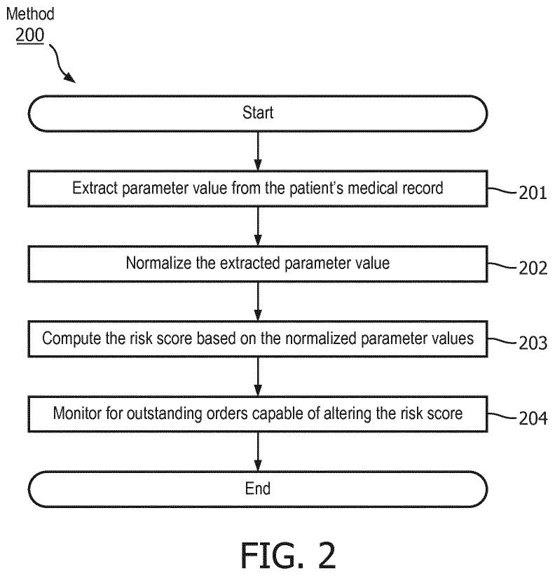

FIG. 2 shows a flow diagram of a method according to an exemplary embodiment.

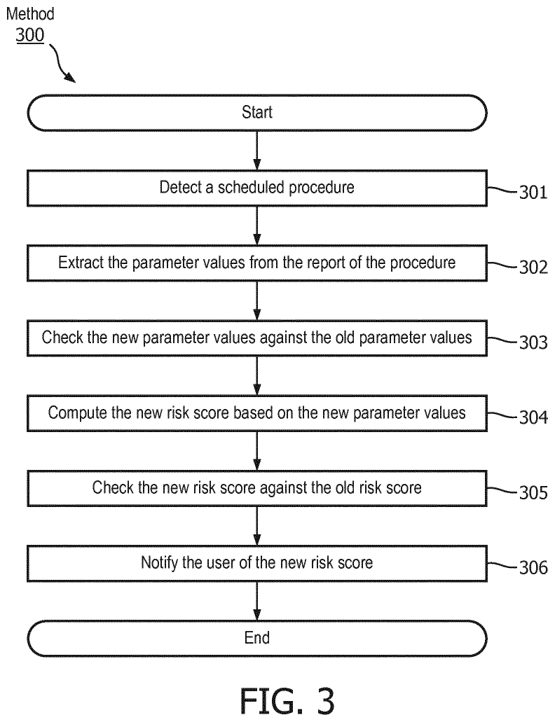

FIG. 3 shows a flow diagram of a method according to an exemplary embodiment.

### DETAILED DESCRIPTION

The exemplary embodiments seek to provide solutions to the above described issues and are aimed to address a problem that is rooted in computer technology. Specifically, real-time alerting of the user to changes to the risk score may avoid the patient undergoing a procedure deemed more dangerous to their health than previous calculated. Among other things, the exemplary embodiments solve the problem of alerting the user by extracting data from a new lab test, calculating a new risk score and transmitting an indication to the user if the risk score has changed. Further, the exemplary embodiments significantly improve productivity and efficiency while reducing the risk of preventable harm to the patient.

The exemplary embodiments may be further understood with reference to the following description and the appended drawings, wherein like elements are referred to with the same reference numerals.

As shown in FIG. 1, a system 100, according to an exemplary embodiment of the present disclosure, is used to perform the exemplary functionalities that were described above. The system 100 comprises a processor 102, a user interface 104, a display 106, and a memory 108. Each of the components of the system 100 may include various hardware implementations. For example, the processor 102 may be hardware component that comprises circuitry necessary to interpret and execute electrical signals fed into the system 100. Examples of processors 102 include central processing units (CPUs), control unit, microprocessor, etc. The circuitry may be implemented as an integrated circuit, an application specific integrated circuit (ASIC), etc. The user interface 104 may be, for example, a keyboard, a mouse, a keypad, a touchscreen, etc. The display 106 may be a liquid crystal display (LCD) device, a light emitting diode (LED) display, an organic LED (OLED) display, a plasma display panel (PDP), etc. Those skilled in the art will understand that the functionalities of the user interface 104 and display 106 may be implemented in a single hardware component. For example, a touchscreen device may be used to implement both the display 106 and the user interface 104. The memory 108 may be any type of semiconductor memory, including volatile and non-volatile memory. Examples of non-volatile memory include flash memory, read only memory (ROM), programmable ROM (PROM), erasable PROM (EPROM) and electrically erasable PROM (EEPROM). Examples of volatile memory include dynamic random-access memory (DRAM), and fast CPU cache memory, which is typically static random-access memory (SRAM).

The memory 108 includes a database 120. The database 120 may store medical records correlating to patients. The medical records may include source documents, such as clinical reports, lab reports, etc. Those of skill in the art will understand that the source documents may be written, oral or a combination of both.

In an exemplary embodiment, the patient may be linked to a patient identifier. The patient identifier may be any type of an identification code, such as a Medical Record Number (MRN) or a Patient Identifier, used to identify the patient. The patient identifiers may also be stored in the database 120. The database 120 may be structured (e.g. in a structured format) in a manner that allows for patient specific queries. It should also be understood that the database 120 may represent a series of databases or other types of storage mechanisms that are distributed throughout system 100 or other interconnected systems.

The processor 102 may be implemented with engines, including, for example, an intelligent processing engine 111, a risk computation engine 112, a scheduling engine 113, a consistency detection engine 114 and a controller engine 115. Each of these engines will be described in greater detail below. Those skilled in the art will understand that the engines 111-115 may be implemented by the processor 102 as, for example, lines of code that are executed by the processor 102, as firmware executed by the processor 102, as a function of the processor 102 being an application specific integrated circuit (ASIC), etc.

As discussed above, a risk model processes one or more parameters. Each of the parameters may be composed of elementary parameters. For example, the epidermal growth factor receptor (eGFR) parameter of the AKI model may be determined by multiple elementary parameters, such as serum creatinine, age, gender and race. For the sake of simplicity, the term “parameter” will be used to refer to both the parameters and the elementary parameters. Furthermore, while the exemplary embodiments are described with reference to the AKI model, those skilled in the art will understand that the exemplary embodiments may be applied to other types of risk models. For example, other models may include mortality models, bleeding models, etc.

The intelligent processing engine 111 searches for and extracts parameter values from a patient's medical record and normalizes the extracted parameter values with respect to the required input format of the risk model. The parameter values may be searched for from specific or predetermined data sources in the medical record. Three exemplary embodiments will be presented below illustrating the searching and extracting process. However, those skilled in the art will understand that other methods may be used to search for and extract the parameter values from the medical record.

In a first exemplary embodiment, the intelligent processing engine 111 may extract the parameter value from free text in the medical record, such as from a clinical report. For example, the intelligent processing engine 111 may extract whether a balloon pump was used on the patient in the past. The extracting may be done by natural language matching techniques. Alternatively, negation scope detection may be used to avoid negative occurrences that may be picked up as positive cues. For example, if the clinical report states that, “there is no report of balloon pump use in the patient,” the negative scope detection will prevent the above recitation of the “balloon pump” from being extracted by the intelligent processing engine 111.

In a second exemplary embodiment, the intelligent processing engine 111 may extract the parameter value from controlled lists of information. For example, the intelligent processing engine 111 may extract a diabetes status from a list of International Statistical Classification of Diseases and Related Health Problems (“ICD”) codes in the medical record or the intelligent processing engine 111 may extract a hypotension status from the medical record. The intelligent processing engine 111 may then match a list of statuses extracted from the medical record against a controlled list. Thus, for example, if a match occurs between the patient's list and the controlled list, such as a ICD code in the patient's list matching a diabetes code in the controlled list, the intelligent processing engine 111 may conclude that the patient is diabetic.

In a third exemplary embodiment, the intelligent processing engine 111 may extract the parameter value from structured documents. For example, the intelligent processing engine 111 may extract the parameter value from a lab report in the patient's medical record. The parameter value may be a measurement or any other information item in a predetermined location of the lab report.

Following the extracting process, the intelligent processing engine 111 may then normalize the extracted parameter values relative to a required input format of the risk model. For example, the diabetes status of the AKI model may require an input of either “yes” or “no”. Similarly, the anemia status of the AKI model may also require an input of either “yes” or “no”. Thus, the normalized parameter values for the diabetes status and the anemia status would be the “yes” or the “no” input. It should be noted that it is customary to use pre-determined threshold values for numerical values. Further, for matching information items and for unnegated detected text fragments, it is customary to set the corresponding status to a “yes”.

The risk computation engine 112 computes a risk score by executing the risk model's mathematical formula. For example, the risk computation engine 112 may retrieve a mathematical formula for the AKI model from the database 120 and input the normalized parameter values into the formula. The normalized parameter values may be a mix of the parameter values normalized by the intelligent processing engine along with parameter values stored on the database 120. Further, the parameter values may be entered manually or automatically. The computation by the risk computation engine 112 may then produce the risk score in the form of, for example, a percentage score, a score category, or any other score. The risk score may correlate to a type of risk model used.

The scheduling engine 113 maintains outstanding orders for the patients. The outstanding orders may be, for example, lab orders, interventional procedures, etc. In an exemplary embodiment, the scheduling engine 113 may retrieve the outstanding orders by transmitting the patient's unique identifier through an Application Programming Interface (API) of a repository that contains the outstanding orders, such as a Picture Archiving Communications System (PACS). Alternatively, the outstanding orders may be retrieved from the database 120.

It should be noted that different outstanding orders may be utilized in different manners. Specifically, outstanding orders such as the lab orders may be regarded for the purposes obtaining updated normalized parameter values in order to update the risk models. Outstanding orders such as the interventional procedures may be regarded for the purposes of using the risk models to determine the risk of the interventional procedure.

The consistency detection engine 114 automatically detects for inconsistencies. The inconsistencies may pertain to those between an old normalized parameter value and a new normalized parameter value or between an old risk score and an updated risk score.

In one possible exemplary embodiment, the consistency detection engine 114 may detect inconsistencies at a parameter level. For example, when two normalized parameter values are present, the consistency detection engine 114 may compare the two parameter values and check for a conflict. In an exemplary embodiment, the diabetes parameter value of the patient may be indicated with a “no” value while a new diabetes parameter value, as determined from a recent lab exam added to the patient's medical record, may indicate that the patient is, in fact, diabetic. The intelligent processing engine 111 may extract the new parameter value and normalize the new parameter value into a “yes” value. The consistency detection engine 114 may then compare the old “no” parameter value with the new “yes” parameter value and determine that the two parameter values conflict with each other.

In another variant, the consistency detection engine 114 may detect inconsistencies at a risk score level or at a risk category level. With the risk score level, the consistency detection engine 114 may compare an old risk score with an updated risk score, as determined by the risk computation engine 112. If the old risk score and the updated risk score are within a permissible range of a pre-determined threshold, the consistency detection engine 114 may determine that there is no conflict. For example, if the old risk score is 9%, the updated risk score is 10% and the predetermined threshold is 5%, then the consistency detection engine 114 would determine that the difference between the old and updated risk scores is within the permissible range and, thus, no conflict exists. In another example, if the old risk score is 9%, the updated risk score is 30%, and the predetermined threshold is 5%, the consistency detection engine 114 would determine that the difference between the old and updated risk scores is not within the permissible range and, thus, there is a conflict.

Alternatively, with the risk category level, the consistency detection engine 114 may compare an old risk category and an updated risk category, as determined by the risk computation engine 112. For example, if the old risk category is “medium” and the updated risk category is “high”, the consistency detection engine 114 may determine that there is a conflict, while if the old risk category and the updated risk category are both “medium”, the consistency detection engine 114 may determine that there is no conflict. It should be noted that a consistency detection engine 114 may be utilized to detect inconsistencies at both the parameter level and the risk score/risk category level, or at only either of the two levels.

The controller engine 115 controls a flow of information between the components of the system 100 and informs the engines 111-114 when new information is available. For example, the controller engine 115 may routinely monitor the scheduling engine 113 to retrieve the patients who have scheduled studies that may affect the risk score, such as a lab test. In an exemplary embodiment, the controller engine 115 may utilize a lookup table that connects each parameter with one or more data sources, such as, for example, the anemia parameter with a complete blood count (CBC) lab test. If the patient has one or more scheduled lab tests, the controller engine may flag the patient as “pending results.” This will be further elaborated upon below.

FIG. 2 shows a method 200, according to an exemplary embodiment, for computing the risk score. In step 201, the intelligent processing engine 111 searches for and extracts the parameter value(s) from the patient's medical record. As discussed above, the extracting may be accomplished by multiple different methods. These methods include, but are not limited to, extracting the parameter value(s) from the free text in the medical report(s) of the patient's medical record, extracting the parameter value(s) from the controlled lists of information in the patient's medical record, and extracting the parameter value(s) from structured documents in the patient's medical record.

In step 202, the intelligent processing engine 111 normalizes the parameter value(s) extracted in step 201. The parameter value(s) are normalized relative to the required input format of the risk model. In step 203, risk computation engine 112 computes the risk score by inputting the normalized parameter value(s) into the mathematical formula of the risk model and executing the formula. The computation by the risk computation engine 112 produces the risk score, which may be, as discussed above, a percentage score, a score category, or any other score that correlates to the type of risk model used.

In step 204, the controller engine 115 routinely monitors the scheduling engine 113 for outstanding orders placed for the patient. The outstanding orders may include, for example, lab orders or interventional procedures scheduled by a physician.

FIG. 3 shows a method 300, according to an exemplary embodiment, for alerting the user of system 100, such as the patient's physician, of any changes to the risk score associated with an interventional procedure. The method 300 will assume the patient's scheduled interventional procedure is a cardiology intervention and the risk model associated with the cardiology intervention is the AKI model. As discussed above, the AKI model predicts the risk of nephropathy caused by contrast use during the cardiology intervention. For example, the risk score produced by the AKI risk model for the patient may be “medium”.

In step 301, the controller engine 115 detects a scheduled lab exam (e.g., complete blood count (CBC) lab test) for the patient. The controller engine 115 may determine that the CBC lab test may potentially change the risk score in the AKI risk model. For example, the CBC may reveal that the patient is anemic, where anemia may be a parameter in the AKI risk model.

In an exemplary embodiment, the controller engine 115 may mark the patient's electronic medical record (EMR) to indicate that the patient has a scheduled lab exam that can potentially change the patient's risk score of the AKI risk model. For example, the patient's EMR may be marked as “pending results”. In one variant, the mark may be seen on the display 106 on the patient's medical record. In another variant, the mark may be seen on the patient lookup list, which may be viewed on the display 106. In a further exemplary embodiment, the patient's physician may be transmitted an alert indicating that the patient has been marked “pending results”. The alert may be transmitted to the physician via text, email, page, application (app) notification, etc.

After the lab exam is concluded and the results of the lab exam are added to the patient's medical record, in step 302, the controller engine 115 engages the intelligent processing engine 111 to extract the parameter values from the lab exam. For example, the controller engine 115 may continue monitoring the patient's medical record and detect that the CBC lab results have been added. Accordingly, the controller engine 115 may engage the intelligent processing engine 111 to extract and normalize the anemia parameter value of the patient. For the purposes of this exemplary embodiment, the intelligent processing engine 111 may determine that that patient is anemic and produce a normalized parameter value of “yes”.

In step 303, the controller engine 115 feeds the normalized parameter value from step 302 into the consistency detection engine 114 to determine if there are inconsistencies. For example, the consistency detection engine 114 may receive the normalized parameter value of “yes” for the anemia value, as determined in step 302. The consistency detection engine 114 may then compare the “yes” value to an old anemia normalized parameter value of, for example, “no”, which would reveal an inconsistency.

In an exemplary embodiment, the user may allow for the new normalized parameter value (e.g., “yes”) to automatically overwrite the old normalized parameter value (e.g., “no”). In an alternative exemplary embodiment, the user may receive an indication asking for permission to overwrite the old normalized parameter value with the new normalized parameter(s) values.

In a further exemplary embodiment, the old normalized parameter value may not exist. That is, the risk score may have been calculated without a certain parameter value. In such an embodiment, the consistency detection engine 114 may determine that there is an inconsistency between an empty parameter value and the new normalized parameter value. Thus, the new normalized parameter value would replace the empty parameter value.

In another exemplary embodiment, if it is determined in step 303 that there is no inconsistency, the controller engine 115 may remove the mark (e.g., results pending) from the patient's medical record. Additionally, the controller engine 115 may notify the patient's physician of the update to the patient's status. Alternatively, if step 303 determines that an inconsistency exists, the method 300 proceed to step 304.

In step 304, the controller engine 115 may engage the risk computation engine 112 to compute a new AKI risk score by updating the anemia value with the new normalized parameter value in the AKI risk model. For example, the anemia value in the AKI risk model may be updated from “no” to “yes”, and the AKI risk model may then be executed. The AKI risk model may then produce a new AKI risk score of, for example, “high”.

In step 305, the controller engine 115 feeds the new AKI risk score from step 304 into the consistency detection engine 114 to determine if there are inconsistencies between the new AKI risk score and the old AKI risk score. For example, the consistency detection engine 114 may compare the old AKI risk score of “medium” and the new AKI risk score of “high” and determine that the two risk scores are inconsistent.

In an exemplary embodiment, if step 305 determines that there is no inconsistency, the controller engine 115 may remove the mark (e.g., results pending) from the patient's medical record. Additionally, the controller engine 115 may notify the patient's physician of the update to the patient's status. Alternatively, if step 303 determines that an inconsistency exists, the method 300 may proceed to step 306.

In step 306, the controller engine 115 notifies the user of the new AKI risk score. For example, the controller engine 115 may transmit an alert indicating that the patient's risk score has changed. The alert may be transmitted to the physician via text, email, page, app notification, etc. Those skilled in the art would understand that the alert may be transmitted to any user of system 100 or to any party deemed pertinent, such as, for example, a cardiologist scheduled to perform the cardiology intervention.

The above exemplary embodiments and methods may be applied to multiple patients simultaneously. For example, the database 120 may contain a patient worklist that lists all patients due for interventional procedures. In an exemplary embodiment, the patient worklist may list patients due for the interventional procedures within a specified timeframe.

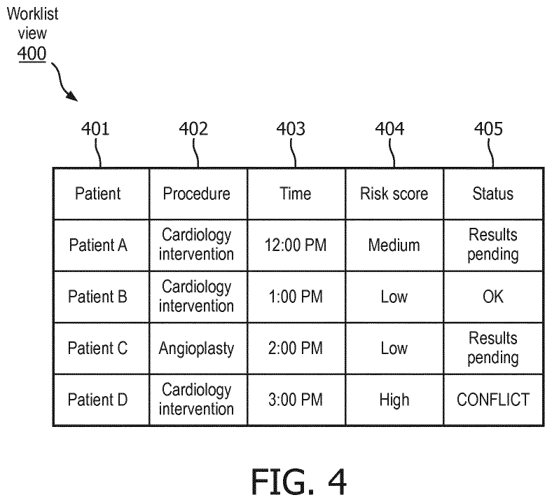

FIG. 4 shows a worklist view 400. In an exemplary embodiment, the worklist view 400 may display, on the display 106, the patient worklist in a user friendly format. In a variant, the worklist view may display the patient 401 (e.g., by name, by MRN, etc.) the interventional procedure scheduled for the patient 402, the time of the scheduled procedure 403, the risk score of the procedure 404, and the status of the patient 405. As shown in FIG. 4, Patient A has a “results pending” status. As discussed above, this may indicate that Patient A has an outstanding order, such as a lab exam, scheduled. Those of skill in the art would understand that the outstanding order may be scheduled before or after the interventional procedure, which gives the user discretion in deciding whether to perform the interventional procedure at the scheduled time or, alternatively, postpone the interventional procedure.

Patient B displays an “OK” status. This may indicate that Patient B has no scheduled outstanding orders. Patient D displays a “Conflict” status. This may indicate that the risk score of Patient D has been updated to “High”. In another variant, the worklist view 400 may employ a different arrangement of categories, including, for example, dates, scheduled outstanding order(s), old risk score(s), etc. Those skilled in the art would understand that the status 405 may be a represented by a string (e.g., descriptive text), integer, color, etc. In a further exemplary embodiment, the user may edit data pertaining to each patient on the patient worklist, such as entering new parameter values, scheduling lab exams, etc.

It is important to note that the worklist view 400 may provide the user, such as the cardiologist, with a tool for effectively and efficiently planning while mitigating potentially harmful procedures.

It will be apparent to those skilled in the art that various modifications may be made to the disclosed exemplary embodiments and methods and alternatives without departing from the spirit or scope of the disclosure. Thus, it is intended that the present disclosure cover the modifications and variations provided that they come within the scope of the appended claims and their equivalents.

It is noted that the claims may include reference signs/numerals in accordance with PCT Rule 6.2(b). However, the present claims should not be considered to be limited to the exemplary embodiments corresponding to the reference signs/numerals.

Those skilled in the art will understand that the above-described exemplary embodiments may be implements in any number of manners, including, as a separate software module, as a combination of hardware and software, etc. For example, the intelligent processing engine 111, the risk computation engine 112, the scheduling engine 113, the consistency detection engine 114 and the controller engine 115 may be programs containing lines of code that, when compiled, may be executed on a processor.

1. A system for alerting a user of a new risk score, comprising:
a processor configured to:
receive a medical report for a patient;
extract medical data of the patient from the medical report;
compute a risk score that is predictive of risk of a certain adverse clinical event based at least in part on the extracted medical data;
compare the risk score to a previous risk score of the patient; and
determine when a change between the risk score and the previous risk score exceeds a predetermined threshold; and
a display that displays an alert when the change exceeds the predetermined threshold.

2. The system of claim 1, wherein the extracted medical data includes a parameter value extracted from free text.

3. The system of claim 1, wherein the extracted medical data includes a parameter value extracted from a controlled list.

4. The system of claim 1, wherein the extracted medical data includes a parameter value extracted from a structured document.

5. (canceled)

6. (canceled)

7. The system of claim 1, wherein the processor utilizes natural language matching techniques to extract the medical data.

8. The system of claim 1, wherein the risk score is one of a percent value or an absolute value, wherein the predetermined threshold is a percent change of the percent value or the absolute value.

9. The system of claim 1, wherein the processor performs the receiving when the patient has an intervention procedure scheduled.

10. The system of claim 1, wherein the processor is further configured to;
detect at least one outstanding order for the patient, wherein the at least one outstanding order may affect the risk score; and
further indicate on the display that the risk score may change.

11. The system of claim 1, wherein the processor is further configured to:
determine a conflict between the medical data extracted from the medical report and previously extracted medical data; and
provide a further alert on the display of the conflict.

12. A method for alerting a user of a new risk score, comprising:
receiving a medical report for a patient;
extracting medical data from the medical report;
computing a risk score that is predictive of risk of a certain adverse clinical event based at least in part on the extracted medical data;
comparing the risk score to a previous risk score of the patient;
determining when a change between the risk score and the previous risk score exceeds a predetermined threshold; and
displaying an alert when the change exceeds the predetermined threshold.

13. (canceled)

14. (canceled)

15. The method of claim 12, further comprising using natural language matching techniques to extract the medical data.

16. The method of claim 12, wherein the extracted medical data includes at least one of a parameter value extracted from free text, a parameter value extracted from a controlled list and a parameter value extracted from a structured document.

17. The method of claim 12, wherein the receiving is conducted when patient has an intervention procedure scheduled.

18. The method of claim 12, further comprising:
detecting at least one outstanding order for the patient, wherein the at least one outstanding order may affect the risk score; and
further indicating on the display that the risk score may change.

19. The method of claim 12, further comprising:
determining a conflict between the medical data extracted from the medical report and previously extracted medical data; and
providing a further alert on the display of the conflict.

20. (canceled)
